(19) United States
(12) Patent Application Publication
(10) Pub. No.: US 2022/0142481 A1
(43) Pub. Date: May 12, 2022

(54) Disposable Sterile Device and Method for Using Disposable Sterile Device

(71) Applicant: Canon U.S.A., Inc., Melville, NY (US)

(72) Inventors: Alexander Altshuler, Cambridge, MA (US); Anderson Thi Mach, Cambridge, MA (US)

(21) Appl. No.: 17/435,394

(22) PCT Filed: Feb. 27, 2020
(86) PCT No.: PCT/US20/20100

§ 371 Date: Sep. 1, 2021

Related U.S. Application Data
(60) Provisional application No. 62/812,891, filed on Mar. 1, 2019.

(51) Int. Cl. A61B 5/00

(52) U.S. Cl. CPC A61B 5/0059; A61B 2560/0285

(57) ABSTRACT

A barrier or cover used for protecting instrumentation in a medical and/or biological setting, and more specifically, a multi-piece apparatus having a multi-use component and a single use component configured to be matted to the multi-use component, wherein the single use component is configured for matting to the multi-use component and shielding the multi-use component.

### CROSS REFERENCE TO RELATED PATENT APPLICATIONS

This application claims priority from U.S. Provisional Patent Application No. 62/812,891 filed on Mar. 1, 2019, in the United States Patent and Trademark Office, the disclosure of which is incorporated herein in its entirety by reference.

### FIELD OF THE DISCLOSURE

The present disclosure relates in general to a disposable device for use in a medical and/or biological setting. Moreover, the subject disclosure relates to disposable device capable of quick and efficient engagement and replacement by an end user, to be used in a sterile environment.

### BACKGROUND OF THE DISCLOSURE

A large number of current medical and biological procedures require a sterile barrier to prevent cross contamination between a patient/sample and a device used for the procedure. For elongated devices the barrier used is usually a polymer film tube connected to the proximal end of non-sterile portion of the device and extending to at least the end of required sterile field enclosing the device. As a new barrier must be deployed for each procedure, ensuring a sterile environment, the ease and expedience of the barriers replacement is germane to efficient work flow.

A first prior art example of a sterile bag/drape for the medical field is taught in U.S. Patent Application Publication No. U.S. No. 2014/0338676 A1, entitled “Medical Drape And Methods Of Covering Equipment With Medical Drapes” to Marinchak. A sterile drape 4 is provided to cover medical equipment 8, 10, 36. The drape 4 may include an expandable element 30 to open the open end of the drape 16. A sheath may also be provided to cover the drape 4 prior to use. The drape 4 may also be held in a container, which may be coupled to the equipment. Furthermore, the drape 4 may include one or more partially deployed positions in which the drape 4 is partially deployed yet the sterile surface of the drape 4 remains unexposed so that the drape 4 may be partially deployed by non-sterile personnel and stored in a non-sterile area.

A second prior art example of sterile bag/drape for the medical field is taught in U.S. Pat. No. 5,792,045, entitled “Sterile Surgical Coupler And Drape” to Adair. An apparatus and method are provided for enclosing a non-sterile camera setup comprising a video camera VC, its trailing cables, and a standard optical connector OC for use of the unsterile camera setup in the sterile environment of an operating room. The apparatus includes a coupler having a first end for attachment to the unsterile camera setup VC, FR, OC and a second end for attachment to a sterile endoscope E. A passageway is formed inside the coupler 10 that extends from the first end to the second end thereof providing an optical pathway whereby an image from the endoscope E may be transmitted to the unsterile camera setup VC, FR, OC. A transparent window is mounted transversely across the passageway between the first and second ends of the coupler providing a sterile barrier there between. In operation, a sterile drape 60 is positioned over the first end of the coupler and extends over the unsterile camera setup VC, FR, OC. The sterile drape 60 is secured between the first and second ends of the coupler by a locking ring which secures the distal end of the drape so that a fluid and airtight seal is formed there between. A sterile disposable wand may be releasably attached to the second end of the coupler for providing support to the unsterile camera setup VC, FR, OC as it is attached to the coupler 10. The wand may then be detached enabling connection of the sterile endoscope E to the second end of the coupler 10.

A third prior art example of a sterile bag/drape for the medical field is taught in U.S. Pat. No. 8,459,266, entitled “Pleated Bag For Interventional Pullback Systems” to Glynn et al. A system is provided that includes a pullback device, catheter, and pleated bag. Pleated bags 115 are used with interventional pullback systems 5, including imaging catheters such as IVUS catheters, as well as for use with other catheters. An exemplary pleated bag 115 of the system may be an elongate, sterile bag, having a plurality of pleats near the distal end, and including an orifice proximal to the pleats designed to allow passage of a catheter into the bag. The above pleated bag system allows for improved imaging catheter procedures at reduced costs.

Despite these advancements in the art, current sterile device deployment methods continue to be cumbersome and sometimes require coordinated efforts of a sterile and non-sterile person to implement. Accordingly, there exists a need in the art for an improved and streamlined device and process for utilizing a disposable sterile device.

### SUMMARY

The present disclosure aims to teach a new disposable sterile device and methods of use thereof, for implementation in medical and biological procedures.

In one embodiment, the subject disclosure teaches a medical apparatus for use in medical or biological procedures, comprising: a multi-use component; and a single use component configured to be removably matted to the multi-use component, wherein the single use component is configured for matting to the multi-use component by a single hand of an end user, and wherein the single use component at least partially shields the multi-use component.

In another embodiment, the subject innovation includes a support bracket for removably bracing the multi-use component.

In further embodiments, the apparatus further comprises an at least partially substantially rigid cover integral with the single use component and configured to at least partially shield an outer surface of the multi-use component, when coupled together, to prevent cross-contamination.

In yet another embodiment, the apparatus further comprises a hand guard configured to prevent contact between the end user and an unshielded portion of the multi-use component. The hand guard may be configured on either the single use component or multi-use component, and perhaps even both.

In another contemplated embodiment, the support bracket of the apparatus is further adapted to assist in engagement and disengagement of the single use component with the multi-use component.

Furthermore, the support bracket is further adapted to hold the single use component and the multi-use component while matted.

In certain embodiment, the apparatus multi-use component comprises at least one optical fiber in communication with a light source and an optical sensor.

In another embodiment, the multi-use component comprises at least one optical fiber in communication with a probe.

Furthermore, an embodiment is envisioned wherein the multi-use component comprises at least one optical fiber for optical signal transmission.

Additionally, the multi-use component may be further adapted to impart rotational motion to the single use component.

In further embodiments, the multi-use component and the single use component are adapted for quick engagement and disengagement.

In yet another embodiment, the subject innovation discloses an apparatus for use in medical or biological procedures, comprising: a multi-use component; a single use component configured to be matted to the multi-use component; and a deployable shield coupled to the single use component, wherein the single use component is configured for matting to the multi-use component by an end user, wherein the single use component at least partially covers the multi-use component, and wherein the deployable shield at least partially shields the multi-use component.

In various iterations, the apparatus further comprises an at least partially substantially rigid cover integral with the single use component and configured to at least partially shield an outer surface of the multi-use component to prevent cross-contamination.

In further variants, the medical apparatus further comprises a hand guard configured to prevent contact between the end user and an unshielded portion of the multi-use component.

In further embodiment, the medical apparatus includes a support bracket, wherein the support bracket is further adapted to assist in engagement and disengagement of the single use component with the multi-use component.

These and other objects, features, and advantages of the present disclosure will become apparent upon reading the following detailed description of exemplary embodiments of the present disclosure, when taken in conjunction with the appended drawings, and provided paragraphs.

### BRIEF DESCRIPTION OF THE DRAWINGS

Further objects, features and advantages of the present disclosure will become apparent from the following detailed description when taken in conjunction with the accompanying figures showing illustrative embodiments of the present disclosure.

FIG. 1 provides a front perspective view of an exemplary sterile device, according to one or more embodiment of the subject apparatus, method or system.

FIG. 2a depicts a front perspective view of an exemplary sterile device, according to one or more embodiment of the subject apparatus, method or system.

FIG. 2b is a front perspective view of an exemplary sterile device engaged with a quick release support bracket, according to one or more embodiment of the subject apparatus, method or system.

FIG. 3 illustrates a front perspective view of an exemplary sterile device, according to one or more embodiment of the subject apparatus, method or system.

FIG. 4 is a cross-sectional view of a quick-release support bracket of an exemplary sterile device, according to one or more embodiment of the subject apparatus, method or system.

FIG. 5 illustrates a front perspective view of an exemplary sterile device, according to one or more embodiment of the subject apparatus, method or system.

FIGS. 6a and 6b depict front perspective views of an exemplary sterile device, according to one or more embodiment of the subject apparatus, method or system.

Throughout the Figures, the same reference numerals and characters, unless otherwise stated, are used to denote like features, elements, components or portions of the illustrated embodiments. In addition, reference numeral(s) including by the designation “′” (e.g. 12′ or 24′) signify secondary elements and/or references of the same nature and/or kind. Moreover, while the subject disclosure will now be described in detail with reference to the Figures, it is done so in connection with the illustrative embodiments. It is intended that changes and modifications can be made to the described embodiments without departing from the true scope and spirit of the subject disclosure as defined by the appended paragraphs.

### DETAILED DESCRIPTION OF THE DISCLOSURE

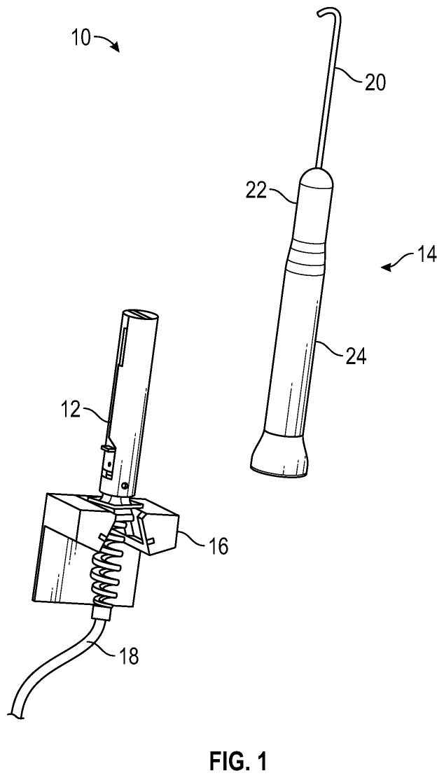

In one embodiment, depicted in FIG. 1, the subject disclosure teaches a medical apparatus 10 for use in medical or biological procedures, wherein the medical apparatus 10 has a multi-use drive 12; a single use handle 14 configured to be removably matted to the multi-use drive 12; and a support bracket 16 for removably housing the multi-use drive 12. The single use handle 14 is configured for matting to the multi-use drive 12 by a single hand of an end user, wherein the single use handle 14 at least partially shields the multi-use drive 12. In the depicted embodiment, the multi-use drive 12 may be held on the quick release support bracket 16, and is in communication with a console (not shown) through a cable 18 extending from the lower portion of the multi-use drive 12. The sterile single use handle 14 includes a probe 20, a finger grip 22, and a drive cover 24 adapted to fit over the multi-use drive 12. A portion of the single use handle 12 is adapted as a hand grip, such that the end user is in contact with the single use handle 12 only.

One or more optical fibers, found in the cable 18, connect the multi-use drive 12 with a light source and/or optical sensor(s) located in the console. The single use handle 14 also comprised of optical fibers, extending through the drive cover 24, for transmitting optical signal(s) to and from the probe 20. These fibers optically connect through the optical connectors with corresponding optical fibers extending to the console when the single use handle 14 is engaged with the multi-use drive 12. In order to image an area around the probe end with OCT, SEE or similar imaging techniques, at least one of the optical fibers needs to rotate. An electric motor 26 contained in the multi-use drive 12 imparts rotational motion onto the optical fiber. The rotational motion is then transmitted to the single use handle 14 and the probe 20 through the optical connector.

Both the single use handle 14 and the multi-use drive 12 are designed to have a mating locking mechanism to engage the two on deployment, to hold them both locked together in operational condition during use, and to disengage after use. A variety of well-known locking schemes may be employed here, such as bayonet connection or a push button spring snap clip, to name a couple.

The support bracket 16 has a quick release designed to accept and securely hold the multi-use drive 12 during storage while still allowing for a quick deployment by a single hand.

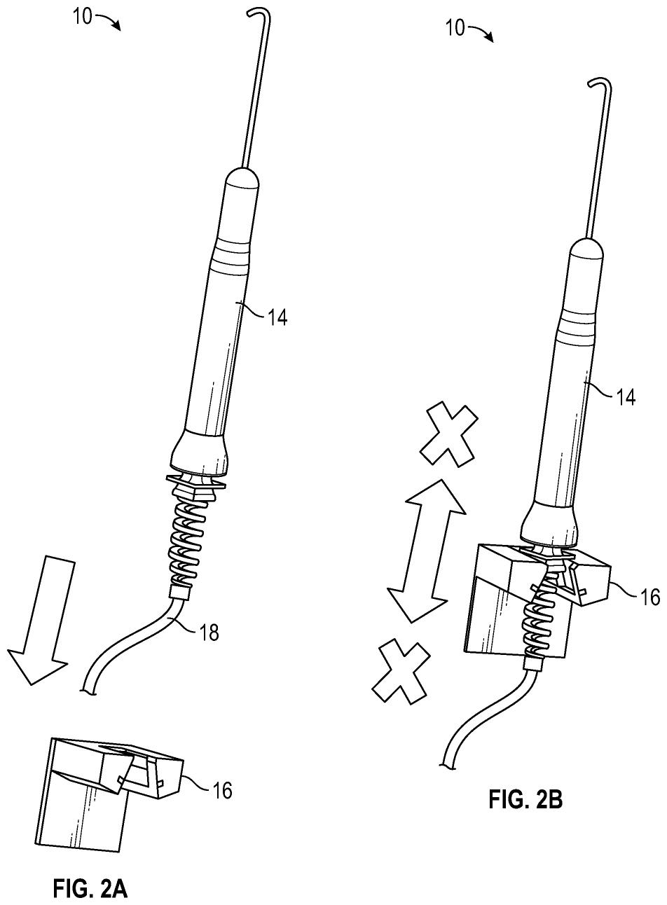

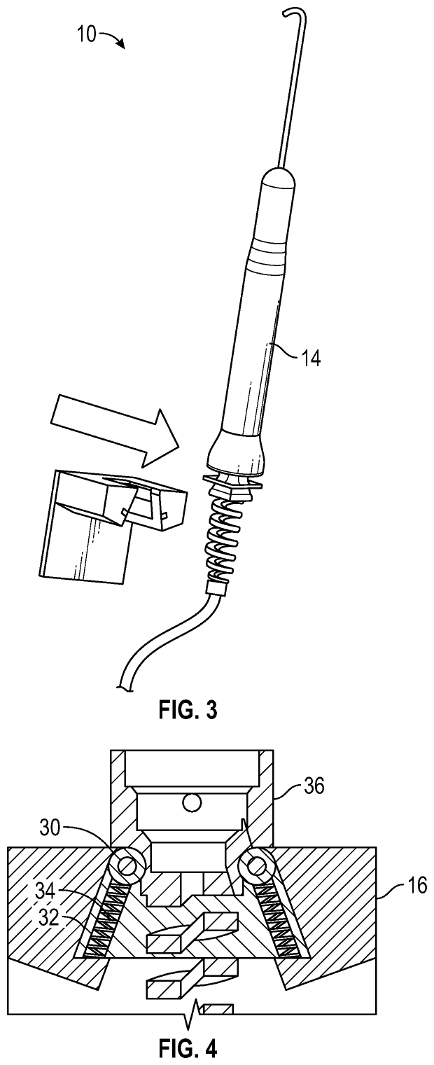

FIGS. 2a and 2b show the single use handle 14 matted with the multi-use drive 12, wherein FIG. 2a shows how the assembled medical device 10 is engaged with the support bracket 16, by advancing the medical device 10 in the direction of the arrow. FIG. 2b depicts the assembled medical apparatus 10 engaged with the support bracket 16, wherein a force applied to the medical device vertically, as shown the arrows, does not disengage the medical device 10 form the support bracket 16. FIG. 3 depicts the combined single use handle 14 engaged with the multi-use drive 12, and how the combined assembled medical device 10 is removed from the quick release support bracket 16 by horizontally disengagement. After use the combined medical apparatus 10 may be installed back onto the quick release bracket 16, as shown in FIG. 2a, wherein the single use handle 14 may be unlocked and disengaged from a multi-use drive 12, allowing for safe disposable of the single use handle 14.

As may be seen from the above description and associated drawings of the sterile disposable single use handle 14, the sterile probe is configured for installation, use, and disposal, all accomplished expediently and completed by a sole sterile operator.

FIG. 4 depicts a cross-sectional view of the preferred embodiment of the quick release support bracket 16. The support bracket 16 is comprised of a pair of opposing cylindrical rollers 30 arranged to roll freely along corresponding inclined guiding surfaces 32 and preloaded in upward direction by springs 34. Referring to FIG. 4, on insertion, a user presses with the bracket engaging portion 36 of the multi-use drive 12 down on the rollers 30 forcing them to roll downwards and sideways along the inclined guiding surfaces 32, to allow the lower portion of multi-use drive 12 to be inserted into the support bracket 16 and locked by the rollers 30 urged upwards by the inclined guiding surface 32. In this position the multi-use drive 12 is positively locked vertically, thus allowing for removal, installation, and/or manipulation of the single-use handle 14. To remove the multi-use drive 12, the user will just have to pull it out horizontally along the length of the rollers 30. The described design affords quick insertion, secure holding, and easy deployment of the multi-use drive 12.

The support bracket 16 may assist in a process of engaging the single use handle 14 with the multi-use drive 12 by restricting vertical and rotational movement of the multi-use drive 12. The support bracket 16 may be further be used for storage of the multi-use drive 12 between uses, for temporary storage of a single use handle 14 engaged with the multi-use drive 12 during a procedure, and for supporting the multi-use drive 12 during cleaning.

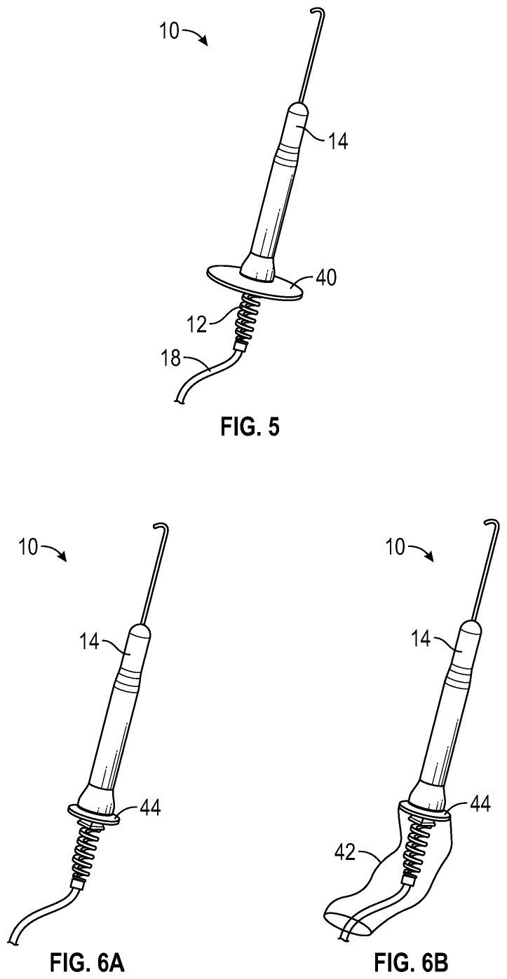

In another embodiment, the single use handle 14 may also comprise a hand guard 40 to prevent inadvertent contact between the end user's hand and the multi-use drive 12 and/or connecting cable 18. As seen in FIG. 5, the hand guard 40 is configured at or near the end of the single use handle 14, so as to deter contact of the multi-use drive 12 by the end user.

In yet another embodiment, detailed in FIGS. 6a and 6b, the single use handle 14 may also incorporate a stored sterile drape 42 that may be additionally deployed over the non-covered portion of the multi-use drive 12 and/or connecting cable 18, if needed. A rigid ring 44 or otherwise shaped feature on the proximal end of the drape 42 may house the deployable drape 42, and assist in its deployment.

As used herein, the term single use component is not limited to a disposable component, but is further meant to include any component which may require removal for cleaning, reconditioning, storage or disposal between uses of the system, wherein the single use component may be used multiple times.

While the above disclosure describes certain illustrative embodiments, the invention is not limited to the above-described embodiments, and the following claims include various modifications and equivalent arrangements within their scope.

1. An apparatus for use in medical or biological procedures, comprising:
a multi-use component; and
a single use component configured to be matted to the multi-use component;
wherein the single use component is configured for matting to the multi-use component by a single hand of an end user, and
wherein the single use component at least partially shields the multi-use component.

2. The apparatus for claim 1, further comprising a support bracket for removably bracing the multi-use component.

3. The apparatus of claim 1, further comprising an at least partially substantially rigid cover integral with the single use component and configured to at least partially shield an outer surface of the multi-use component to prevent cross-contamination.

4. The apparatus of claim 1, further comprising a hand guard configured to prevent contact between the end user and an unshielded portion of the multi-use component.

5. The apparatus of claim 2, wherein the support bracket is further adapted to assist in engagement and disengagement of the single use component with the multi-use component.

6. The apparatus of claim 2 wherein the support bracket is further adapted to hold the single use component and the multi-use component while matted.

7. The apparatus of claim 1, wherein the multi-use component comprises at least one optical fiber in communication with a light source and an optical sensor.

8. The apparatus of claim 1, wherein the multi-use component comprises at least one optical fiber in communication with a probe.

9. The apparatus of claim 1, wherein the multi-use component comprises at least one optical fiber for optical signal transmission.

10. The apparatus of claim 1, wherein the multi-use component is further adapted to impart rotational motion to the single use component.

11. The apparatus of claim 1, wherein the multi-use component and the single use component are adapted for quick engagement and disengagement.

12. A system for supporting a removable device comprising:
a support body with at least one inclined surface;
an at least one roller adapted to move along the at least one inclined surface, the roller having an axis; and
a connector portion adapted for insertion into the support body,
wherein the connector portion comprises an engagement feature adapted to at least partially enfold the at least one roller on engagement,
wherein the support body has an at least one opening adapted for removal of the removable device in a direction of the roller axis.

13. The system of claim 11 further comprising an at least one spring element to bias roller into engage position.

14. The system of claim 11 further comprising at least two substantially symmetric inclined surfaces and at least two rollers each adapted to move along a corresponding inclined surface.

15. An apparatus for use in medical or biological procedures, comprising:
a multi-use component;
a single use component configured to be matted to the multi-use component; and
a deployable shield coupled to the single use component,
wherein the single use component is configured for matting to the multi-use component by an end user,
wherein the single use component at least partially covers the multi-use component, and
wherein the deployable shield at least partially shields the multi-use component.

16. The apparatus of claim 15, further comprising an at least partially substantially rigid cover integral with the single use component and configured to at least partially shield an outer surface of the multi-use component to prevent cross-contamination.

17. The apparatus of claim 15, further comprising a hand guard configured to prevent contact between the end user and an unshielded portion of the multi-use component.

18. The apparatus of claim 15, further comprising a support bracket for removably bracing the multi-use component.

19. The apparatus of claim 18, wherein the support bracket is further adapted to assist in engagement and disengagement of the single use component with the multi-use component.

20. The apparatus of claim 16, wherein the deployable shield is configured for one time use.

21. The apparatus of claim 16, wherein the deployable shield is stored within the single use component.
